(19) United States
(12) Patent Application Publication
(10) Pub. No.: US 2024/0108483 A1
(43) Pub. Date: Apr. 4, 2024

(54) LUMEN STABILIZING DEVICE

(71) Applicant: UNITED STATES ENDOSCOPY GROUP, INC., Mentor, OH (US)

(72) Inventors: Christopher J. Kaye, Berea, OH (US); Keith R. John, Chardon, OH (US)

(21) Appl. No.: 18/534,318

(22) Filed: Dec. 8, 2023

Related U.S. Application Data
(63) Continuation of application No. 16/895,799, filed on Jun. 8, 2020.
(60) Provisional application No. 62/858,101, filed on Jun. 6, 2019.

(51) Int. Cl. A61F 2/88

(52) U.S. Cl. CPC A61F 2/88; A61F 2002/825

(57) ABSTRACT

A device for stabilizing a lumen in an open position having a core with a longitudinal axis along the length of the core, and support elements that can be bristles, fibers, or discs, secured to the core, extending radially outward from the core, and having ends that can contact and hold open a lumen, while maintaining the position of the device in the lumen.

### CROSS-REFERENCE TO RELATED APPLICATIONS

The present application is a continuation of U.S. application Ser. No. 16/895,799, filed Jun. 8, 2020, which claims the benefit of and priority to U.S. Provisional Patent Application No. 62/858,101, filed on Jun. 6, 2019, the entire disclosures of which are incorporated herein by reference.

### FIELD OF THE INVENTION

The various embodiments relate to a lumen stabilizing device in general and more particularly to a device having multiple discrete contact points extending radially outward from a center core to contact the inner wall of a lumen to keep the device anchored in place. The lumen can be located within the gastro-intestinal tract or in the cardiovascular system such as a blood vessel, or a lumen in any other system, including within a human body or an artificial lumen.

### BACKGROUND INFORMATION

Conventional stents are hollow tubular structures, that can have cutouts or other openings in the wall of the stent, used to hold a lumen open. Typically, the tube frame of the stent exerts a force against the interior wall of a lumen to anchor the stent in place. Traditional tubular stents can also result in fluid buildup between the outer wall of the stent and the obstruction which the stent is intended to alleviate. This reduction in flow creates less disruptions to biofilm growth.

Improvements to conventional stents may be made to reduce forces applied to the ends of the device by the lumen and thereby prevent tissue from extending into the lumen path. Reducing the biofilm growth also reduces the formation of an anchoring point which can collect and trap debris.

### SUMMARY OF THE INVENTION

In an exemplary embodiment, a device for stabilizing a lumen in an open position has an elongate central core with a longitudinal axis extending along the length of the core, and support elements secured to the core, extending along at least a portion of the core, extending in a radially outward direction from the core, where each support element is attached at one end to the core and has a free end for contacting the interior wall of a lumen. The support elements can be bristles or fibers. The bristles and/or fibers can form a spiral around the core or can be grouped in discrete bunches of bristles and/or fibers. The bristles can have varying lengths. In another exemplary embodiment, the support elements can be fins or disc-like frames, having one or more of a variety of geometries. In another exemplary embodiment, the support elements can have an umbrella-like structure, with arms or prongs extending out from a center region. The support elements permit fluid flow from one end of the device to another while providing support to the lumen. The center core can be comprised of twisted wire, or polymer and/or carbon fiber filaments, which can be twisted, woven or in a straight strand configuration. In addition, the center core can be created by a profile extrusion that can take on an I-beam-like geometry, either straight or in a twisted configuration. Further, the center support structure can emanate from a base that is either flat, curved or with a U-channel geometry.

These and other aspects of the exemplary embodiments will become apparent from the following detailed description, taken in conjunction with the accompanying drawings, illustrating by way of example the principles of the various exemplary embodiments.

### BRIEF DESCRIPTION OF THE DRAWINGS

In order to facilitate a fuller understanding of the exemplary embodiments, reference is now made to the appended drawings. These drawings should not be construed as limiting but are intended to be exemplary only.

FIG. 1 depicts a lumen stabilizing device with a center core and bristles extending in a radially outward direction from the core, according to an exemplary embodiment.

FIG. 2 depicts a lumen stabilizing device with a center core and bristles extending in a radially outward direction from the core, with cutouts in the bristle pattern, according to an exemplary embodiment.

FIG. 3 depicts a lumen stabilizing device with a center core and bristles extending in a radially outward direction from the core, with cutouts in the bristle pattern, according to an exemplary embodiment.

FIG. 4 depicts a lumen stabilizing device with a center core and bristles extending in a radially outward direction from the core, with greater portions of the bristles cutout than in the embodiment depicted in FIG. 3.

FIG. 5A depicts a lumen stabilizing device with a center core and bristles with flattened ends extending in a radially outward direction from the core, according to an exemplary embodiment.

FIG. 5B depicts a close-up view of bristles with flattened ends according to the exemplary embodiment of FIG. 5A.

FIG. 6A depicts a lumen stabilizing device with a center core and bristles with flared ends extending in a radially outward direction from the core, according to an exemplary embodiment.

FIG. 6B depicts a close-up view of bristles with flared ends according to the exemplary embodiment of FIG. 6A.

FIGS. 7A-7C depict exemplary embodiments of lumen stabilizing devices with a center core and bristles extending in a radially outward direction from the core, the bristles having various overlapping patterns.

FIG. 8 depicts a schematic of a lumen stabilizing device in a lumen, according to an exemplary embodiment.

FIG. 9 depicts a lumen stabilizing device with a center core and fiber bundles extending in a radially outward direction from the core, at discrete intervals along the core, according to an exemplary embodiment.

FIG. 10 depicts a lumen stabilizing device with a center core and bristle bundles with cutout sections extending in a radially outward direction from the core, at discrete intervals along the core, according to an exemplary embodiment.

FIG. 11 depicts a lumen stabilizing device with a center core and bristles having various heights extending in a radially outward direction from the core, according to an exemplary embodiment.

FIG. 12A depicts a lumen stabilizing device with a center core and bristles extending in a radially outward direction from the core, according to an exemplary embodiment.

FIG. 12B depicts the lumen stabilizing device of FIG. 12A in a reservoir submerged in a fluid, according to an exemplary embodiment.

FIG. 13A depicts a fin-like frame for placement on a center core of a lumen stabilizing device, according to an exemplary embodiment.

FIG. 13B depicts a lumen stabilizing device with a plurality of fin-like frames on a center core, according to an exemplary embodiment.

FIG. 14A depicts a fin-like frame with shaped end pieces for placement on a center core of a lumen stabilizing device, according to an exemplary embodiment.

FIG. 14B depicts a lumen stabilizing device with a plurality of fin-like frames with shaped end pieces on a center core, according to an exemplary embodiment.

FIG. 15A depicts a star-shaped frame for placement on a center core of a lumen stabilizing device, according to an exemplary embodiment.

FIG. 15B depicts a lumen stabilizing device with a plurality of star-shaped frames as depicted in FIG. 15A on a center core, according to an exemplary embodiment.

FIG. 15C depicts an end view of the lumen stabilizing device with a plurality of star-shaped frames depicted in FIG. 15B.

FIG. 16A depicts an end view of a fin-like frame on a center core with an end geometry inside a lumen, according to an exemplary embodiment.

FIG. 16B depicts an end view of a frame on a center core inside a lumen, according to an exemplary embodiment.

FIG. 17 depicts an arm geometry that can be positioned on a center core, with flared ends, according to an exemplary embodiment.

FIG. 18 depicts an arm geometry that can be positioned on a center core, according to an exemplary embodiment.

FIG. 19 depicts a schematic of a lumen stabilizing device with an arm geometry on a center core, according to an exemplary embodiment.

FIG. 20 depicts a schematic of a lumen stabilizing device with an arm geometry on a center core, according to an exemplary embodiment.

FIG. 21 depicts a schematic of a lumen stabilizing device with an arm geometry on a center core, according to an exemplary embodiment.

FIG. 22 depicts a schematic of a lumen stabilizing device with an arm geometry on a center core, according to an exemplary embodiment.

FIG. 23 depicts a schematic of a lumen stabilizing device with an arm geometry on a center core, according to an exemplary embodiment.

FIG. 24 depicts a lumen stabilizing device with an arm geometry on a center core, according to an exemplary embodiment.

FIG. 25 depicts a lumen stabilizing device with an arm geometry on a center core, according to an exemplary embodiment.

FIG. 26A depicts a lumen stabilizing device with a center beam and support members on opposing sides of the beam, coextruded and twisted, according to an exemplary embodiment.

FIG. 26B depicts a cross sectional view of the device depicted in FIG. 26A.

FIG. 26C depicts a perspective view of a lumen stabilizing device with a center beam and support members on opposing sides of the beam, coextruded and twisted, and having a defined center core, according to an exemplary embodiment.

FIG. 26D depicts a cross sectional view of the device depicted in FIG. 26C.

FIG. 26E depicts a cross sectional view of a lumen stabilizing device with four support members extending from a center core, according to an exemplary embodiment.

FIG. 26F depicts a perspective view of a lumen stabilizing device with three support members extending from a center core, according to an exemplary embodiment.

FIG. 27 depicts a lumen stabilizing device with a curved substrate and bristles extending from the substrate, according to an exemplary embodiment.

FIG. 28 depicts a cross-section view of a lumen stabilizing device placed in a lumen, according to the exemplary embodiment illustrated in FIG. 27.

FIG. 29 depicts a side view of the lumen stabilizing device in a lumen according to the exemplary embodiment illustrated in FIG. 27.

FIG. 30A depicts a core with a hollow center and support members extending in a radially outward direction from the tube, according to an exemplary embodiment.

FIG. 30B depicts the core with a hollow center and support members of FIG. 30A, with a solid center core extending through the hollow center, according to an exemplary embodiment.

FIG. 31 depicts a lumen stabilizing device with an atraumatic tip, according to an exemplary embodiment.

FIG. 32 depicts a delivery system for a lumen stabilizing device, according to an exemplary embodiment.

FIG. 33A depicts a lumen stabilizing device with a plurality of discs with fenestrations on a center core, according to an exemplary embodiment.

FIG. 33B depicts an end view of the lumen stabilizing device of FIG. 33A, according to an exemplary embodiment.

FIGS. 33C-D depict exemplary embodiments of oval-shaped disc frames having fenestrations for placement on a center core as illustrated in FIG. 33A.

FIG. 33E depicts an exemplary embodiment of a disc having curvilinear segments, for placement on a center core as illustrated in FIG. 33A.

### DETAILED DESCRIPTION OF EMBODIMENTS

The following description is intended to convey a thorough understanding of the embodiments by providing various embodiments and details involving a lumen stabilizing device. There is a need for a device that can remain in place in a lumen to hold the lumen open and with improved fluid flow to permit fluid to flow past and/or through the device, without causing tissue to extend into the lumen path, and without encouraging biofilm to grow on the device. Various embodiments include a center core and elements extending radially outward from the center core, such that the ends can contact the inner wall on a lumen. The radially extending elements can be bristles of varying height and stiffness and spaced in various configurations across the center core. The radially extending elements can also be plates or discs, with arms and various shaped ends for contacting the lumen wall. Various additional embodiments include extruded lumen stabilizing devices having a central support structure with portions of the extrusion contacting the interior lumen wall and providing a force to hold the lumen open. In other embodiments, there can be an extruded or molded substrate with bristles extending therefrom, or an extruded or injection molded geometry which possesses a center core.

It is understood, however, that the invention is not limited to these specific embodiments and details, which are exemplary only. It is further understood that one possessing ordinary skill in the art, in light of known devices, systems and methods, would appreciate the use of the invention for its intended purposes and its benefits in any number of alternative embodiments.

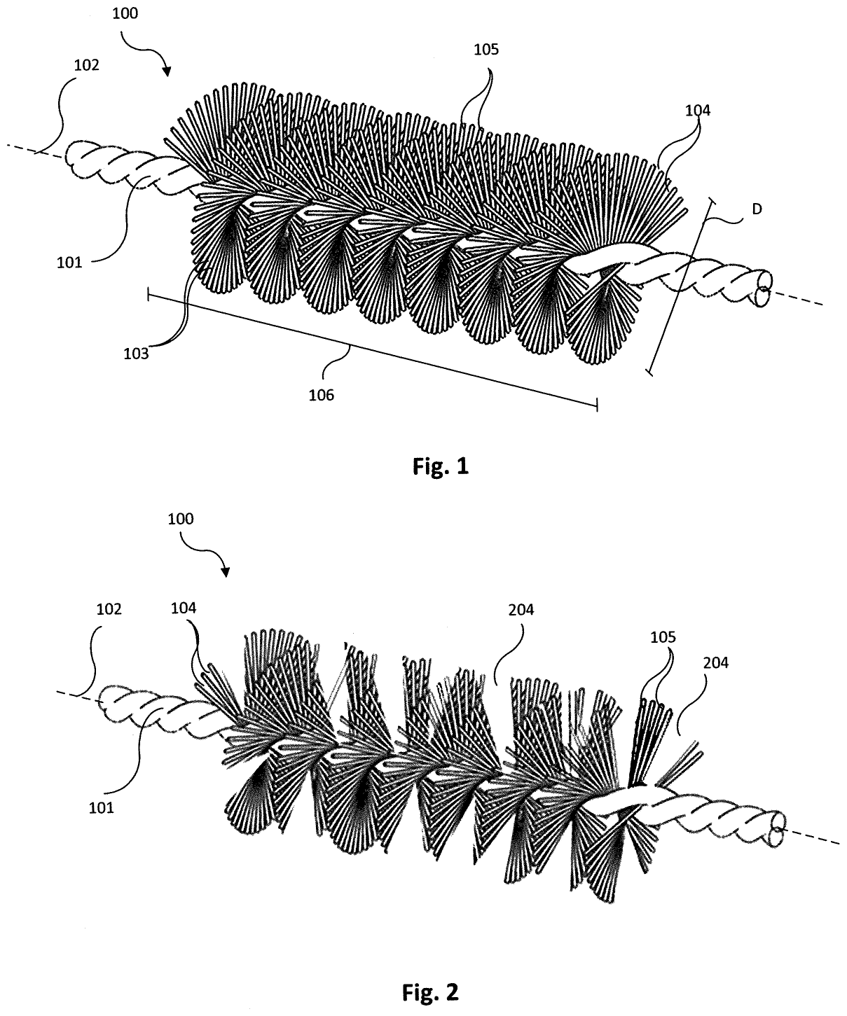

Referring now to FIGS. 1-4, exemplary embodiments of a lumen stabilizing device with a center core and bristles emanating from the center core are illustrated. FIG. 1 illustrates a device 100 having a center core 101 that can be a twisted wire, having a longitudinal axis 102 extending along the length and through the center of the center core 101, with radially extending elements extending outward from the core in a spiral pattern. The center core can be made of filaments, such as wire threads, glass fibers, carbon fibers, or polymer fibers, or a combination of these materials that are either in a straight strand configuration or are twisted or woven. The center core can also be extruded and made of a polymer, such as a Nylon. The center core can be solid or it can have a lumen extending therethrough. A lumen extending through the center core can reduce the weight of the device, and can be used for a guidewire or other guide tool to extend through when implanting the device or be merely present to improve the efficiency of the manufacturing process, A lumen extending through the center core should be of a diameter small enough to not encourage flow of fluid or particles through it. The radially extending elements can be bristles 103 extending outward from the core in a rotary pattern to form a spiral 106 extending outwardly from a length of the center core. The length of the spiral can be the entire length of the core 101 or there can be portions of the center core extending along the axis 102 that do not have bristles. The bristles 103 can be individual fibers extending from the core. The fibers can be spaced apart from each other, and can be spaced apart from each other evenly, as illustrated in FIG. 1. The bristle spacing 104 can be of a density that permits fluid flow from one end of the device to the other, in the direction of the center core longitudinal axis 102 when the device is placed in a lumen, such as a portion of the gastro-intestinal tract or a blood vessel. The bristles can have a diameter ranging from 0.001 inch to 0.035 inch diameter.

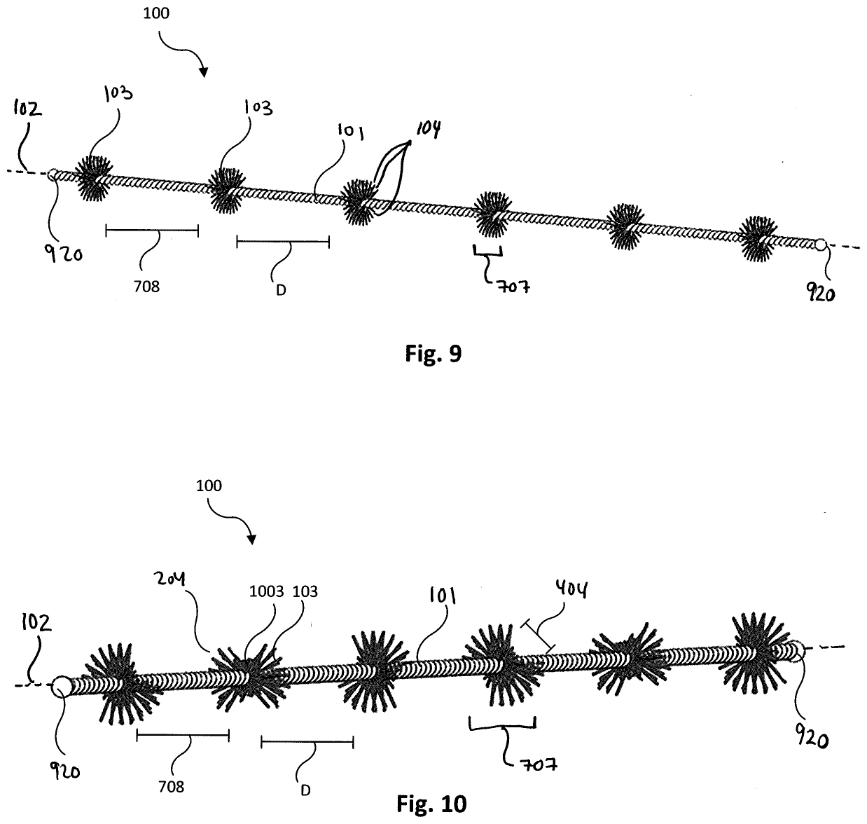

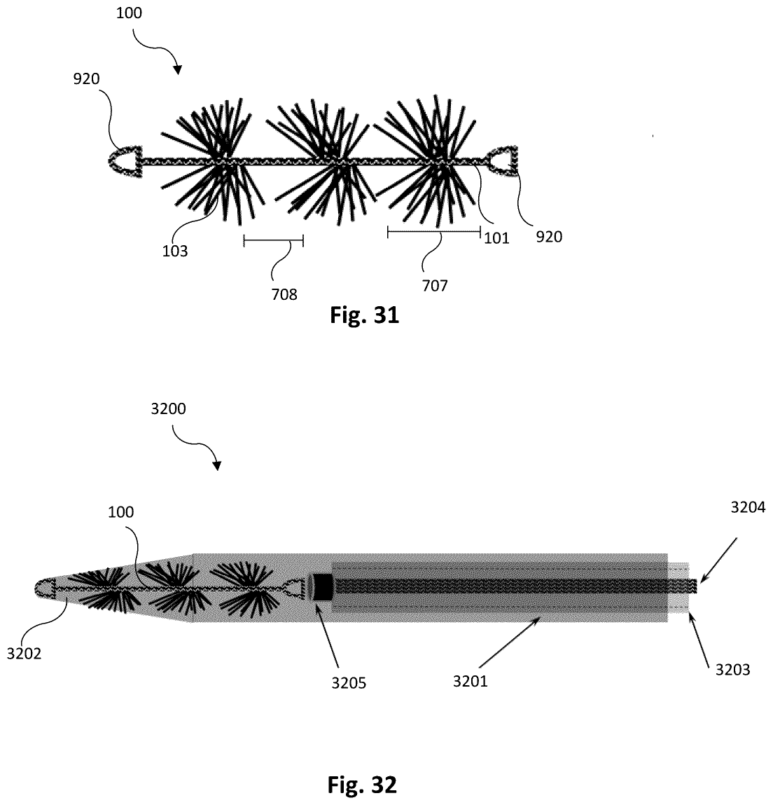

The bristle ends 105 provide many discrete contact points against the tissue wall to keep the device anchored in place. Having numerous discrete contact points distributes the forces between the device and the lumen more uniformly when compared to the distribution of forces that are directed to the two ends of a continuous open tube-style stent. The greater the number of bristle ends (i.e., contact points), the greater the distribution of the forces between the device and the lumen. Further, the bristle ends 105 provide many discrete areas of contact against the lumen wall that inhibits the undesirable formation of a continuous biofilm. Biofilm growth must create a scaffolding structure to bridge the gaps when such a device having numerous contact points on the lumen wall, such as the plurality of bristles 103 illustrated in FIGS. 1-4 is in the lumen. The growth of a biofilm scaffolding structure takes longer to develop than a biofilm layer on a traditional tubular stent with a long continuous surface. As the biofilm scaffold grows, the fluid flow from one end of the device to the other can wash out the biofilm growth, delaying the build-up of a scaffolding structure. In implant locations where biofilm is not desired, this increases the effective life of the lumen stabilizing device. Initial tissue growth response upon implantation of the device can result in the formation of a natural lumen. The proximal end of the device can have a grasping feature 920 (see FIG. 9) which a user can use to grasp the end of the device to remove or reposition it. The grasping feature can be a ball or loop or other shape. There can also be a grasping feature on the distal end (see FIG. 9). The grasping feature 920 can also be a loop or other atraumatic tip (see FIG. 31).

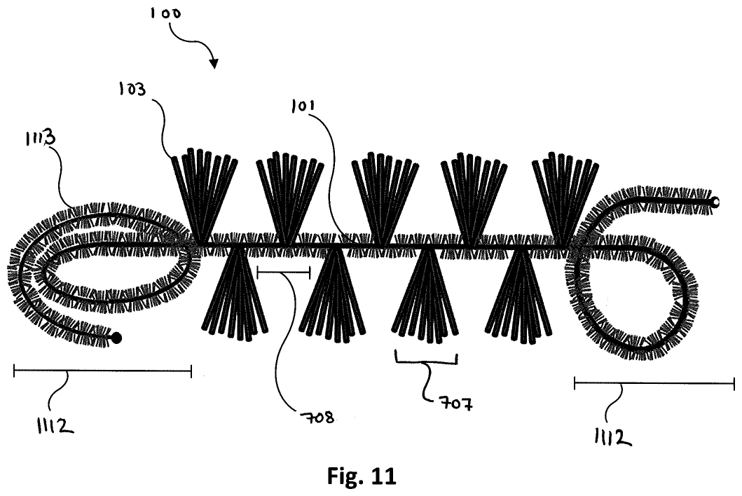

The device 100 can be made of a bio-absorbable material such as those derived from lactide, glycolide, trimethylene, carbonate, p-dioxanone and ε-caprolactone. The device can be made of shape memory material such as Nitinol, to allow a variety of final shapes and/or configurations. For example, the ends of the center core of the device can be coiled (see FIG. 11). The lumen stabilizing device can also be drug-eluting. The fibers and/or core that are fabricated with a hydrophilic material such as Nylon can absorb water, facilitating the release of a drug.

The spiral shape of the bristles can have a constant outer diameter D along the length of the device 100. In another embodiment, the spiral shape can have a varying outer diameter D; for example, the outer diameter can decrease along the length of the device in a conical shape. Varying the outer diameter of the lumen stabilizing device provides a different flow path for the fluid as it moves through the lumen from one end of the device to the other. Varying the outer diameter of the lumen stabilizing device can also be used as another way to adjust the anchoring force when the device is positioned in a lumen.

FIG. 2 illustrates an exemplary embodiment of a lumen stabilizing device 100 with a center core 101 that can be a twisted wire, having a longitudinal axis extending along the length and through the center of the center core 101, with radially extending elements 103 extending outward from the core in a spiral pattern. The radially extending elements can be bristles extending outward from the core in a rotary pattern to form a spiral extending outwardly from a length of the core. The length of the spiral can be the entire length of the core 101 or there can be portions of the core extending along the axis 102 that do not have bristles. The bristles 103 can be individual fibers extending from the core. The fibers can have sections cut out from the spiral shape, as illustrated in FIG. 2. The cutouts 204 can be spaced apart evenly, or the spacing of the cutouts can vary. The cutouts can be of the same size, or of various sizes. The cutouts 204 are one way to adjust the fluid flow in the direction of the axis of the center core, and the remaining bristles provide support to hold the lumen open. The size of the cutouts can vary depending on the lumen, the device is to be placed in, and the viscosity of the fluid that passes through that lumen.

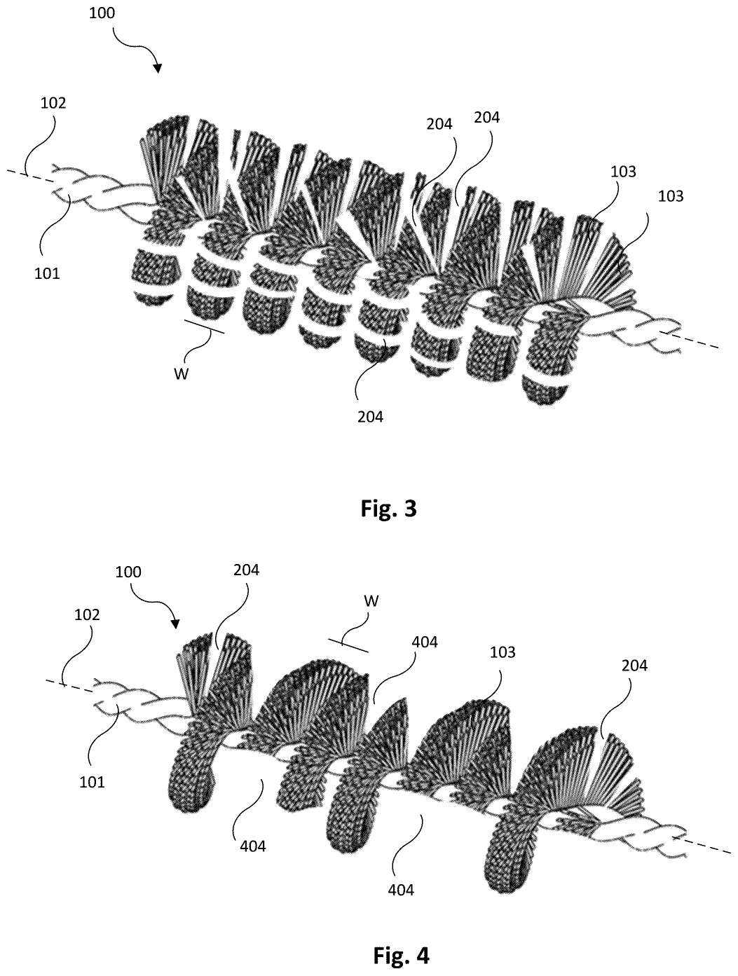

FIG. 3 illustrates an exemplary embodiment of a lumen stabilizing device 100 with a center core 101 that can be a twisted wire, having a longitudinal axis extending along the length and through the center of the center core 101, with radially extending elements 103 extending outward from the core in a spiral pattern. The radially extending elements can be bristles extending outward from the center core in a rotary pattern to form a spiral extending outwardly from a length of the center core. The bristles can be in bundles. The spiral can have a width of a plurality of bristles extending from the center core such that the spiral of bristles “width” W of more than one bristle. In another embodiment, the spiral can be made of bundles of bristles extending outward from the center core. The length of the spiral can be the entire length of the center core 101 or there can be portions of the center core extending along the axis 102 that do not have bristles extending outwardly therefrom. The bristles 103 can be individual fibers extending from the core. As with the other embodiments described herein, the fibers can have sections cut out from the spiral shape, as illustrated in FIG. 3. The cutouts 204 can be spaced apart evenly, or the spacing of the cutouts can vary. The cutouts can be of the same size, or of various sizes. The cutouts 204 result in greater fluid flow in the direction of the axis of the center core than having a spiral pattern alone, and the remaining bristles provide support to hold the lumen open. Further, the cutouts 204 are optional and there can be a lumen stabilizing device 100 with bristles grouped into a width W as in FIG. 3 without having cutouts, that permits fluid flow through the bristles in a direction of the longitudinal axis 102 of the center core 101.

FIG. 4 illustrates an exemplary embodiment of a lumen stabilizing device 100 with a center core 101 that can be a twisted wire, having a longitudinal axis extending along the length and through the center of the center core 101, with radially extending elements 103 extending outward from the core in a spiral pattern. The radially extending elements can be bristles extending outward from the core in a rotary pattern to form a spiral extending outwardly from a length of the core. As with the embodiment illustrated in FIG. 3, the bristles can have a width to them so that the spiral of bristles emanating from the core has a “width” W of more than one bristle. The length of the spiral can be the entire length of the core 101 or there can be portions of the core extending along the axis 102 that do not have bristles extending outwardly therefrom. The bristles 103 can be individual fibers extending from the core. As with the other embodiments described herein, the fibers can have sections cut out from the spiral shape, as illustrated in FIG. 3. The cutouts 204 can be spaced apart evenly, or the spacing of the cutouts can vary. The cutouts can be of the same size, or of various sizes. The cutouts illustrated in FIG. 4 have larger sections of the bristles removed than the device illustrated in FIG. 3. As illustrated in FIG. 4, the size of the cutout sections can vary. There can be smaller sections 204 of bristles cutout and larger sections 404 of bristles cutout. Varying the size and number of the cutouts 204, 404 allows the volume of the fluid flow to be adjusted. The more cutouts there are, the greater the volume of fluid that can flow from one end of the device to the other. The remaining bristles provide support to hold the lumen open.

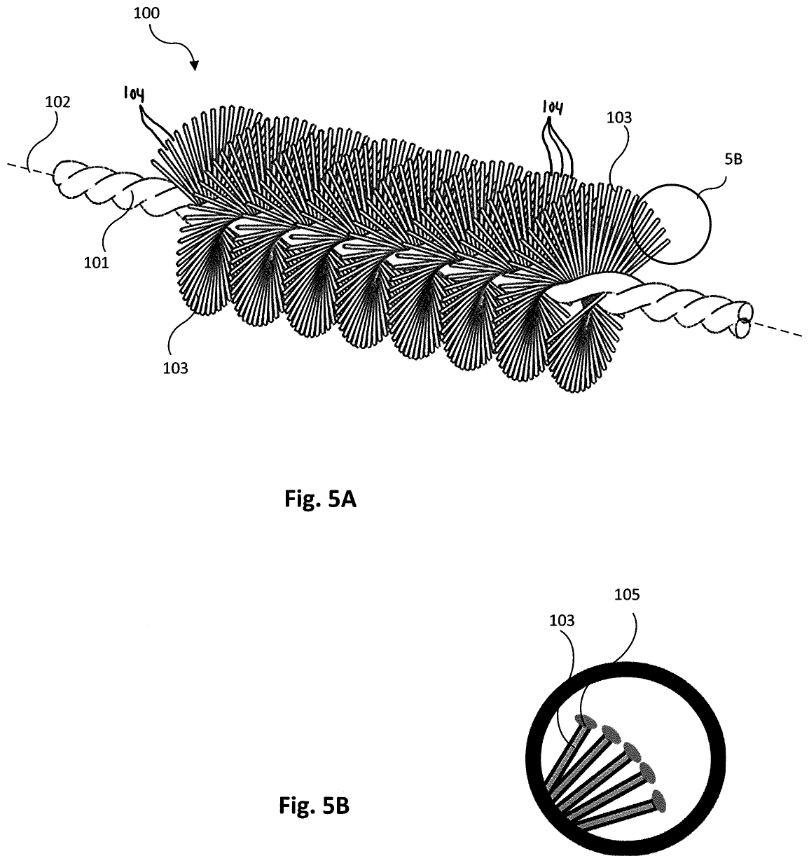

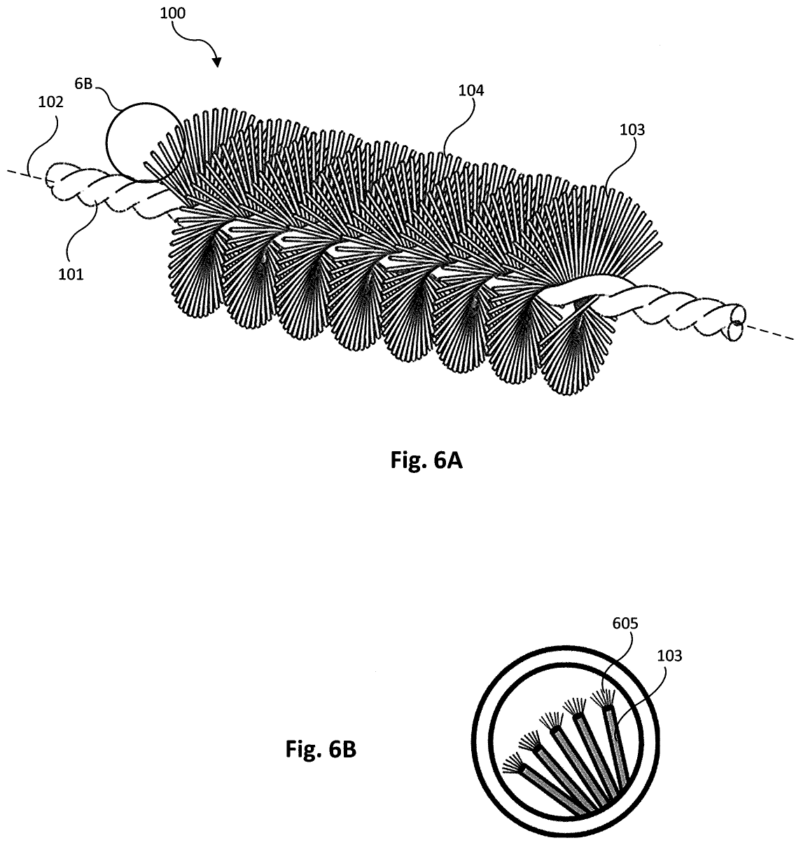

Referring now to FIGS. 5A-6B, a lumen stabilizing device 100 having alternatively shaped atraumatic bristle ends 105 is illustrated. FIGS. 5A and 5B illustrate a device 100 having a center core 101 that can be a twisted wire, having a longitudinal axis extending along the length and through the center of the center core 101, with radially extending elements 103 extending outward from the core in a spiral pattern. The radially extending elements can be bristles extending outward from the core in a rotary pattern to form a spiral extending outwardly from a length of the core. The length of the spiral can be the entire length of the core 101 or there can be portions of the core extending along the axis 102 without bristles. The bristles 103 can be individual fibers extending from the core. The fibers can be spaced apart from each other, and can be spaced apart from each other evenly, as illustrated in FIG. 1. The bristle spacing 104 can be of a density that permits fluid flow from one end of the device to the other, in the direction of the center core longitudinal axis 102 when the device is placed in a lumen. The ends 105 of the bristles can be flat. FIG. 5B illustrates a close-up of the flattened ends 105. The flattened ends can add surface area to each bristle, which act to anchor the device within the lumen. This can result in fewer fibers that would inhibit flow, while still supporting the lumen.

FIGS. 6A and 6B illustrate another exemplary embodiment of an alternatively shaped bristle end. In FIGS. 6A and 6B, the ends 605 of individual bristles are split into many finer fibers 605, to create a flared end. The flared ends add surface area to the bristle ends used to anchor the lumen stabilizing device in the lumen. This can result in fewer fibers that would inhibit flow, while still supporting the lumen.

Referring again to FIGS. 5A-6B, the ends of the bristles can be any combination of regularly cut ends, flattened ends, and/or flared ends. Further, the ends can take on other configurations, including any configuration that increases the surface area of contact between the end of the bristles and the interior wall of a lumen, to act as an anchor. The ends of the bristles can be atraumatic and/or can enhance the grip of the bristles on the interior lumen wall.

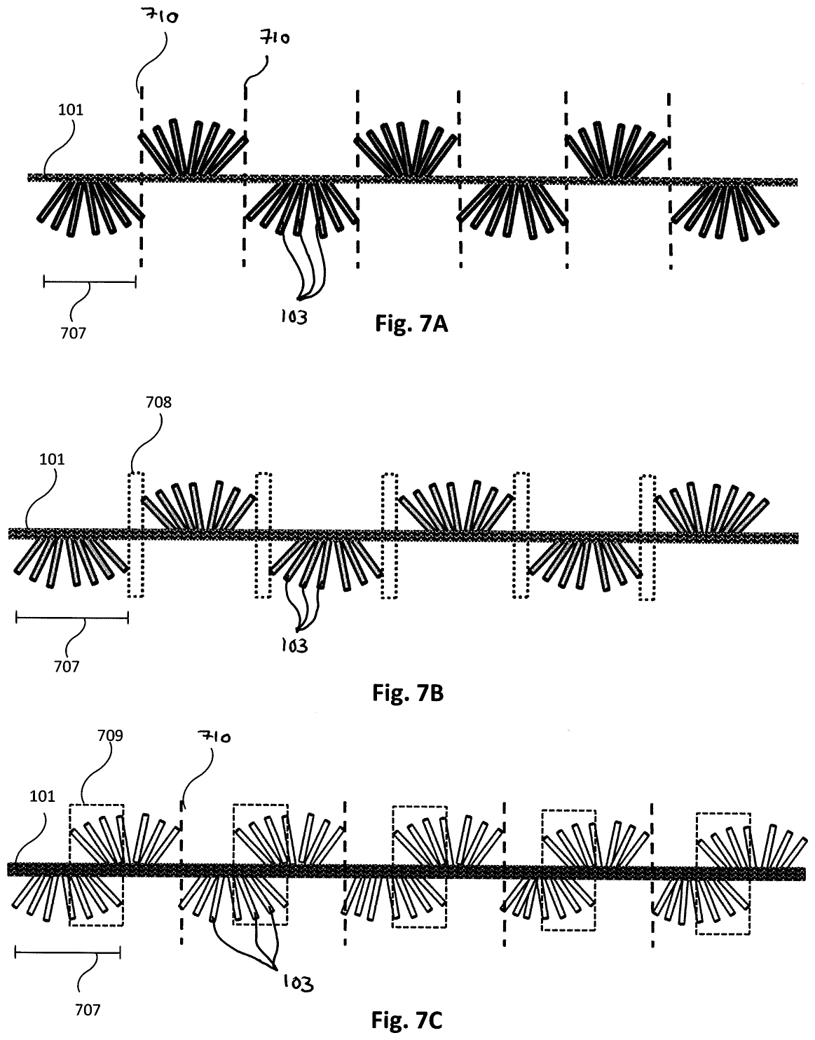

Referring now to FIGS. 7A-7C, schematics of exemplary embodiments of the arrangement of the bristles of a lumen stabilizing device made of a center core 101 that is a twisted wire, and bristles 103, as described above, are illustrated. FIG. 7A illustrates a twisted brush wire configuration device having alternating wire brush groupings 707. The wire brush groupings alternate which side of the twisted wire they extend radially outward from. In FIG. 7A, the direction in which the wire brush groupings 707 appear to extend alternates by 180 degrees. However, the wire brush groupings can alternate by any number of degrees, such as 45 degrees, 90 degrees, or 135 degrees. The wire bristles also can rotate in a spiral form, such as that illustrated in FIGS. 3-6, in each wire brush grouping. FIG. 7A illustrates the wire brush groupings where there is no overlap between the distal ends of the bristles in one group with the distal ends of the bristles in any other group. The alignment of the bristle groups in FIG. 7A is indicated by lines 710.

The available flow volume provided by a lumen stabilizing device varies depending on the dimensions of the center core and the bristles and can be chosen based on the size of the lumen it is to be positioned within. A lumen stabilizing device for placement within a biliary duct lumen, having a center core and bristles extending radially outward from the core such as that illustrated in FIG. 7A, can have a center core diameter ranging from 0.5 mm to 1.5 mm, or from 0.6 mm to 0.8 mm, or from 0.6 mm to 0.8 mm. The center core can have a length ranging from 90 mm to 110 mm, or from 99 mm to 105 mm, or from 99 to 105 mm. The length of the bristles that contact the interior lumen wall to support the lumen wall and prevent migration of the device can have a length ranging from 2.5 mm to 3.5 mm, or ranging from 2.7 mm to 3.3 mm, or ranging from 2.9 mm to 3.1 mm. The bristle fiber diameter can vary, and can range from 0.05 mm to 1.50 mm, or from 0.06 mm to 0.1 mm, or it can range from 0.07 mm to 0.1 mm. These exemplary measurements provide an overall dimension of the device, from the end of one bristle to the end of another bristle that extends from the center core in a direction 180 degrees opposite the first bristle, ranging from 3.0 mm to 3.5 mm, or ranging from 3.2 mm to 3.4 mm, or ranging from 3.2 mm to 3.3 mm. In exemplary embodiments of the lumen stabilizing device where the bristles are grouped together in bundles that occupy a somewhat conical shape, with the apex of the “cone” connected to the center core, the bristle bundles can occupy a volume having a bristle height ranging from 1.5 mm to 1.8 mm, or from 1.6 mm to 1.7 mm. The length of the bristles can be longer in embodiments having an extruded base, where the bristles must extend across a diameter of the lumen to reach and make contact with the interior lumen wall, for example the embodiment illustrated in FIG. 27. The “cone” base, defined by the ends of the bristles that contact the interior lumen wall, can have a radius of 3.0 mm to 3.3 mm, or ranging from 3.1 mm to 3.3 mm, or from 3.1 mm to 2.3 mm. The bunches of bristles can be separated by gaps along the center core, and the gaps can range in distance from 4.0 mm to 10.0 mm, or ranging from 6.0 mm to 8.5 mm, or from 6.2 mm to 6.5 mm. In an exemplary embodiment, the bristles can have a length of 3.0 mm and a diameter of 0.1 mm; the center core can have a diameter of 0.7 mm and a length of 102 mm, and the device can have a distance spanning the diameter of a lumen of 3.4 mm. The dimensions provided herein are exemplary for a lumen stabilizing device that is to be positioned within a biliary tract lumen; the dimensions and geometries can vary beyond the ranges provided for positioning in the biliary tract or within a blood vessel or lumen in yet another system in a body.

FIG. 7B illustrates a schematic having gaps 708 between the bristle bundles in the center core. FIG. 7C illustrates a schematic where the bristle bundles overlap each other along the twisted wire core, identified by the overlapping region 709. In FIG. 7C, there are no gaps along the wire core 101 between the bristle bundles as in FIG. 7B. However, any combination of gaps 708 and overlapping regions 709 can be used to create a lumen stabilizing device. Each gap 708 is a section along the length of the center core that has no bristles. In an embodiment having bristle bundles, the gap can be the length along the center core that has no bristle bundle or other support members extending radially outward form the center core. This is distinct from the cutouts 404 which are sections where the bristles are shorter than the immediately adjacent bristles. Cutouts can occur in any type of bristle formation.

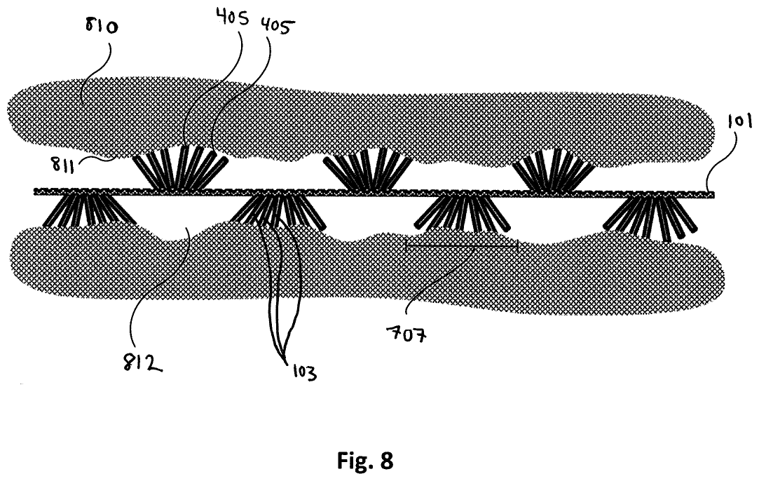

Referring now to FIG. 8, a schematic of an exemplary embodiment of a device in a lumen 812 is illustrated. The device in FIG. 8 has a center core and bristle bundles 707 extending radially outward from the core. The ends of each bristle contact the interior lumen wall 811 to provide support to hold the lumen open and to anchor the device in place, preventing migration of the device once implanted. The device in FIG. 8 illustrates bristle bundles that have no overlap between bundles, such as that illustrated in FIG. 7A, however, any of the exemplary embodiments described herein can be implanted in a lumen. In FIG. 8, the device is inside a lumen 812, and the ends 105 of the bristles 103 are in contact with the interior lumen wall 811 and provide support to hold the lumen open while maintaining the position of the device in the lumen 812.

FIG. 9 illustrates a device 100 having a center core 101 that can be a twisted wire, having a longitudinal axis extending along the length and through the center of the center core 101, with radially extending elements 103 extending outward from the core. The radially extending elements can be bristles extending outward from the core in a rotary pattern extending outwardly from particular segments of the core. The bristle segments can each be separated by a gap 708 having a distance D, along the length of the core 101. The distance between the gaps can be the same in between each bristle segment, or the distance between each bristle segment can vary. The bristles 103 can be individual fibers extending from the core. The fibers can be spaced apart from each other, and can be spaced apart from each other evenly, as illustrated in FIG. 1. The bristle spacing 104 can be of a density that permits fluid flow from one end of the device to the other, in the direction of the center core longitudinal axis 102 when the device is placed in a lumen. The large gaps 708 between the discrete bristle bundles 707 allow fluid flow to occur in the direction of the longitudinal axis 102 of the center core 101 and provide support to the lumen to hold the lumen open. The gaps can vary in size based on the area of the anatomy and the overall length of the lumen in which the device is being placed.

As discrete fibers, the bristles 103 can flex out of the way as independent groups to allow items to slowly pass from one end of the device to the other. The longer bristles can provide anchoring support for the device and the shorter bristles can flex and bend to permit items to pass through with the fluid flow, thereby preventing debris from accumulating on surfaces of the device and the surfaces of the tissue. The flexibility of the bristle fibers can vary. Mixing softer, thinner fibers with more rigid fibers can vary the force on the surrounding tissue along the axial length of the device.

FIG. 10 illustrates a device 100 having a center core 101 that can be a twisted wire or filament, having a longitudinal axis extending along the length and through the center of the center core 101, with radially extending elements 103, 1003 extending outward from the core. The radially extending elements can be bristles extending outward from the core in a rotary pattern extending outwardly from particular segments of the core. The bristle segments can each be separated by a gap 708 having a distance D, along the length of the core 101. The distance between the gaps can be the same in between each bristle segment, or it can vary. The length of the spiral can be the entire length of the core 101 or there can be portions of the core extending along the axis 102 that do not have bristles extending outwardly therefrom. The bristles 103, 1003 can be individual fibers extending from the core. As illustrated in FIG. 10, the bristles can have varying lengths, within the same bristle segment. Longer bristles 103 provide support against the lumen wall. The shorter bristles 1003 permit additional fluid flow in the direction of the longitudinal axis 102. The stiffness of the bristles can vary with the length of the bristles. The bristle spacing 204, 404 can be of a density that permits fluid flow from one end of the device to the other, in the direction of the center core longitudinal axis 102 when the device is placed in a lumen. The bristle bundles 707 can have cutout sections 204, 404. The cutouts can be cut out of the longer bristles, the shorter bristles, and/or both. The large gaps 708 between the discrete bristle bundles allow fluid flow to occur in the direction of the longitudinal axis 102 of the center core 101 and provide support to the lumen to hold the lumen open.

Referring now to FIG. 11, an exemplary embodiment of a device 100 having bristles extending radially outward from the core along the entire length of the core is illustrated. The center core 101 of the device in FIG. 11 can also have helically coiled ends 1112. The helically coiled ends provide stability to the device to maintain its position once placed in a lumen, and also provide support to hold the lumen open. As illustrated in FIG. 11, the device can have bundles of long emanating bristles, according to any of the exemplary embodiments described herein. The discrete fibers can flex out of the way independently from the flex of each of the other fibers, allowing items to pass slowly from one end of the device to the other. The remaining portions of the center core 101 can have wire bristles 1003 extending radially outward from the core that are shorter than the long emanating bristles 103 of the bundles 707. The shorter wire bristles can be trimmed to the desired length.

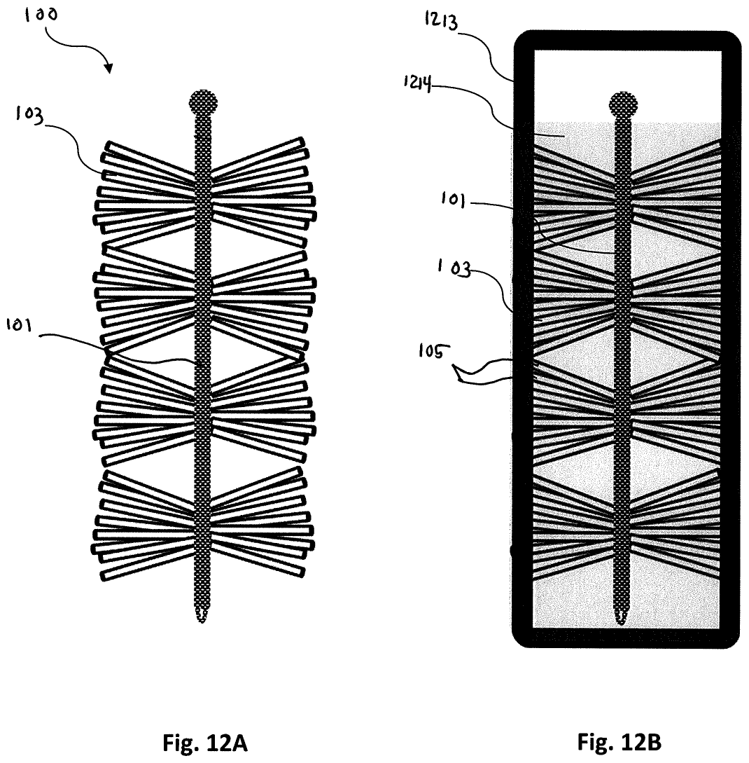

Referring now to FIGS. 12A and 12B, an exemplary embodiment of a lumen stabilizing device 100 and the device submerged in a reservoir 1213 having a fluid 1214 inside is illustrated. The device can be any of the embodiments described herein. The fluid 1214 in the reservoir 1213 can be to administer a lubricious coating, such as a hydrophilic coating. The reservoir is sized such that the bristle ends 103 contact the interior wall 1215 of the reservoir. Insertion of the device 100 into the reservoir 1213 such that the device is submerged in the lubricious coating fluid causes the coating to be applied to the outer surface of the bristles and the center core of the device. The bristle ends 103 that contact the interior wall do not receive the lubricious coating, ensuring that they can serve as anchors for the device in the lumen. This hydrophilic coating along the length of the bristles assists with fluid flow through the length of the device, while the absence of the hydrophilic coating on the bristle ends assists in anchoring the bristles in the lumen. The disclosure herein is not limited to a fluid hydrophilic coating, but the hydrophilic coating can be applied to the device by any conventional means.

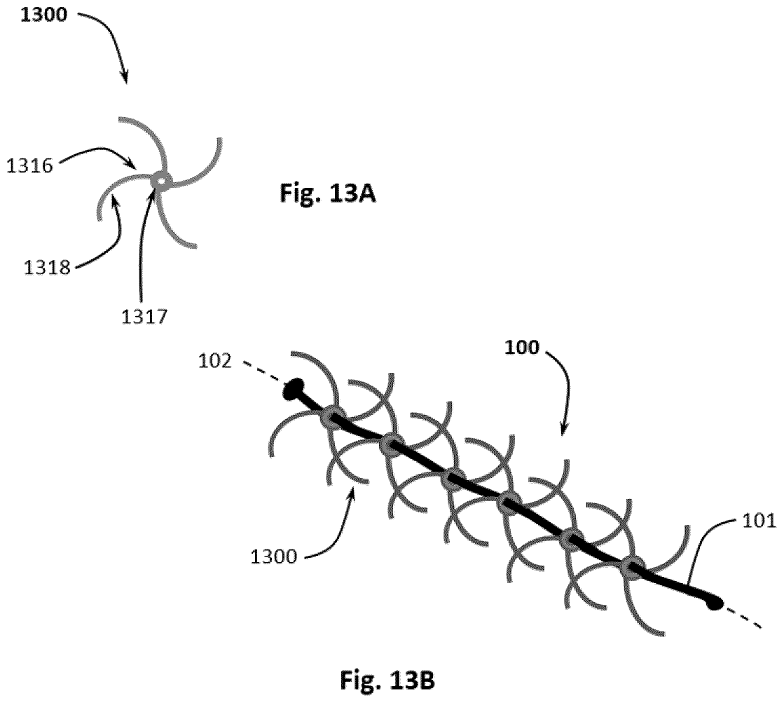

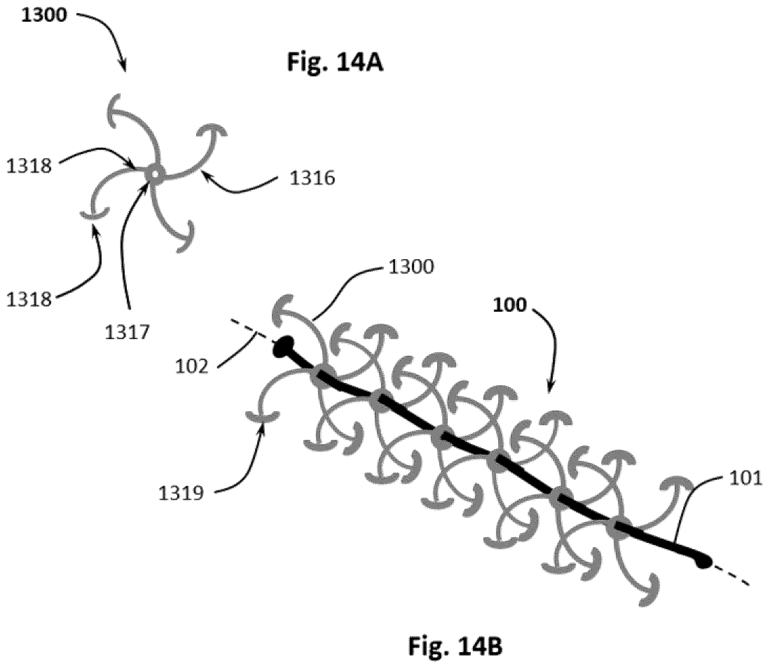

Referring now to FIGS. 13A and 13B, a lumen stabilizing device 100 having fin-like frames 1300 on a center core 101 is illustrated. FIG. 13A illustrates an exemplary embodiment of a fin-like frame 1300. The fin-like frame can have a center region 1316 with a center opening 1317, and a plurality of fins 1318 extending radially outward from the center region 1316. The fin-like frames can be loaded onto the center core 101, and the fins 1318 act as a scaffolding to support the lumen the device is inserted into, maintain the position of the device 100, and permit fluid flow along a longitudinal axis 102 of the center core. The fin-like frames 1300 can be individually molded, cut out, or 3D printed. The fins can be made of a polymer (semi-rigid or elastomeric), and/or metal, and/or a composite material such as glass or carbon fibers. The core can be made of a twisted wire and/or glass fibers or the core can be made of an extruded or injection molded polymer such as HDPE, silicone, TPE, or PTFE.

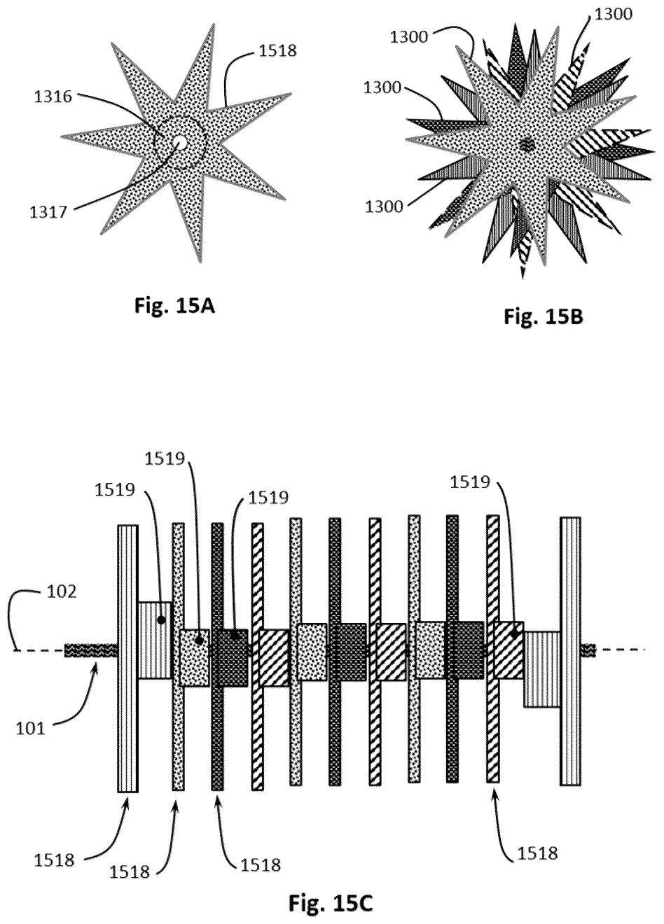

Miniature components anchored to the center core, open areas can be augmented with a variety of physical properties such as tensile strength and flexural modulus, with or without the use of bristles. By creating a device with a center core and then adding numerous discrete elements as shown in FIG. 13, it should be noted that rather than having all the discrete elements the same, that mixing different geometries and material properties can be advantageous. Stiffer members may be necessary to support and immobilize the device in the lumen, whereas softer, more flexible elements can provide some support, but also flex and deform in response to elevated flow pressures that often occur in the fluid channel. In an exemplary embodiment, the relatively rigid star-shaped discs 1300 (as in FIGS. 15A-15C) can be used to provide support to the lumen, and relatively thin fibers such as bristles 103 (as in FIG. 1) or fin-like frames 1300, 1400 can be used to flex and deform in response to the elevated flow fibers. For example, 2-10 star-shaped discs can be used, and the remaining members can be the more flexible fins or bristles. In another example, there can be 3-8, or 3-5 star-shaped discs, and the remaining members can be the more flexible fins or bristles.

Referring now to FIGS. 14A and 14B, a lumen stabilizing device 100 having fin-like frames 1300 on a center core 101 is illustrated. FIG. 14A illustrates an exemplary embodiment of a fin-like frame 1300. The fin-like frame can have a center region 1316 with a center opening 1317, and a plurality of fins 1318 extending radially outward from the center region 1316. The ends 1319 of the fins 1318 can have additional geometries 1319. FIG. 14A illustrates a curved shape geometry, but the geometry can be any shape that increases the contact surface area of the fin on the interior lumen wall. The fin-like frames can be loaded onto the center core 101, and the fins 1318 act as a scaffolding to support the lumen the device is inserted into, maintain the position of the device 100, and permit fluid flow along a longitudinal axis 102 of the center core. The fin-like frames 1300 can be individually molded, cut out, or 3D printed.

Referring now to FIGS. 15A-15C, schematics of a lumen stabilizing device 100 having star-tipped polygon-shaped discs 1300 on a center core 101 are illustrated. FIG. 15A illustrates an exemplary embodiment of a disc 1300. The disc can have a center region 1316 with a center opening 1317, and a plurality of star tips 1518 extending radially outward from the center region 1316. The discs can be loaded onto the center core 101, as illustrated in FIGS. 15B and 15C. The star tips 1518 act as a scaffolding to support the lumen the device is inserted into and maintain the position of the device. The spaces between the star tips on each disc permit fluid flow in the direction of a longitudinal axis 102 of the center core. The discs 1300 can be individually molded, cut out, or 3D printed. The discs can have a disc extension 1519 in a center region extending outward from one side of the disc, which provides an additional volume to the disc. As illustrated in FIG. 15B, the disc extension acts as a spacer between the discs. It should be noted that for one skilled in the art that small extruded tubes cut to the appropriate length or washers can be positioned between the discs to control the space between the discs. FIG. 15B illustrates a side view schematic of a device 100 having star-tipped discs. FIG. 15C illustrates an end view schematic of the device in FIG. 15B.

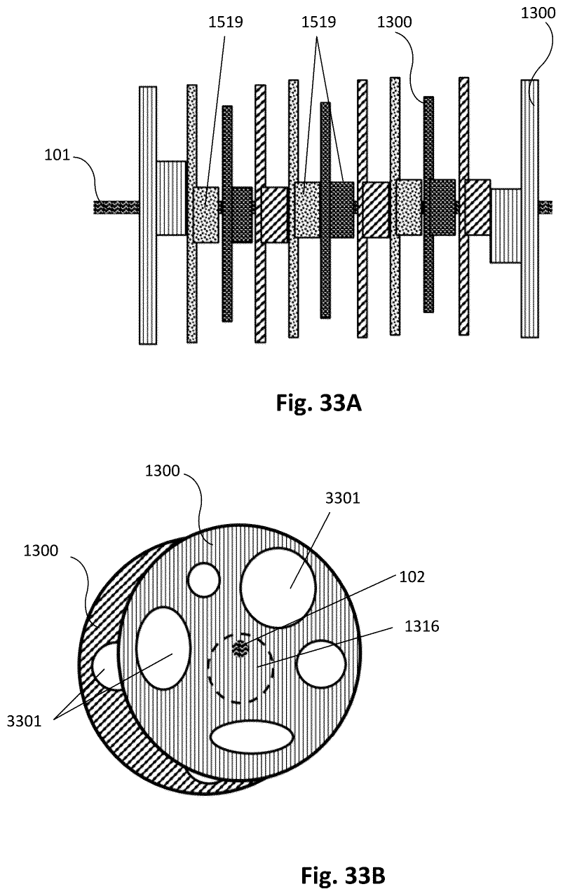

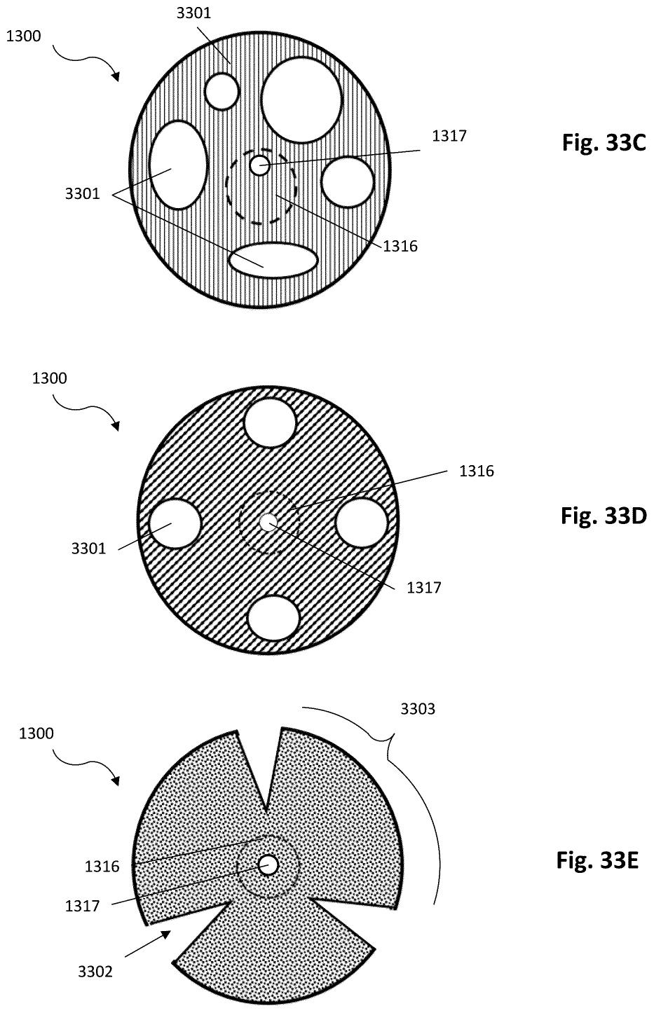

Referring now to FIGS. 33A-33E, schematics of a lumen stabilizing device 100 having discs 1300 on a center core 101, according to an exemplary embodiment, are illustrated. Discs 1300 can be circular or oval shaped, and can have oval shaped fenestrations 3301 to permit fluid flow through the disc. FIG. 33A illustrates a side view of an arrangement of the discs 1300 on a center core 101, and FIG. 33B illustrates and end view of FIG. 33A. FIGS. 33C and 33D illustrate schematics of discs with fenestrations 3301 cut out from the disc. The positioning, shape, and size of the fenestrations 3301 on the discs 1300 can vary, as illustrated in FIGS. 33C and 33D. FIG. 33E illustrates another exemplary embodiment of a disc 1300 having curvilinear segments 3303 separated by cut-outs 3302. The discs can each also have an opening 1316 in a center portion 1316 (shown in FIGS. 33C-33D). The discs are loaded onto the center core to act as scaffolding to support the lumen and maintain the position of the lumen stabilizing device. The discs can be individually molded, cut out, or 3-D printed. The discs can also include a disc extension 1519 to maintain the spacing between the discs when positioned on the center core. The extensions 1519 can be of varying sizes to vary the space between the discs on the core, or they can be the same size. An extension on a disc can be in the center portion 1316 of the disc.

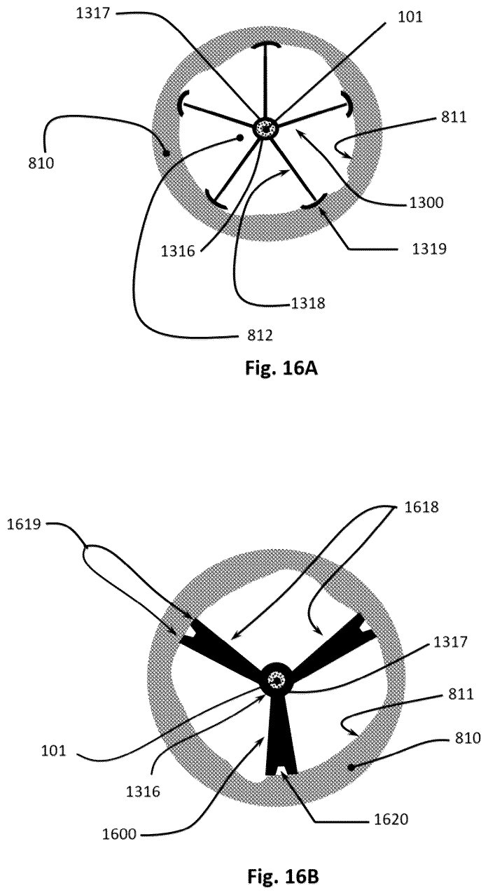

Referring now to FIGS. 16A and 16B, additional frame geometries that can be used to anchor a device and hold a lumen 812 open are illustrated. The frames described herein can be used to support the lumen and maintain the position of the device when implanted. A plurality of discs such as that illustrated in FIG. 16A can be positioned on a center core similar to that illustrated in FIGS. 13B and 14B. The geometry of the disc 1300 in FIG. 16A can have arms or fins 1318 that extend radially outward from a center portion 1316, and the center portion 1316 can have an opening 1317 for positioning on the center core 101. The ends 1319 of the fins in FIG. 16A have an additional geometry in a curved shape, similar to that illustrated in FIGS. 14A and 14B. The discs in FIGS. 16A and 16B can be used with the twisted wire bristles to support a lumen and maintain the position of the device once it has been implanted, while resisting forces from peristaltic waves in the lumen.

FIG. 16B illustrates another exemplary geometry of a disc 1600 having a plurality of arms 1618 extending radially outward from a center region 1316, in a “propeller” configuration. The center region has an opening 1317 for the disc to be positioned on a center core 101, such as that illustrated in FIG. 14B. In FIG. 16B, each arm 1618 has a concave end surface 1620. With this geometry, each arm 1618 has two anchor points 1619 that contact the interior lumen wall.

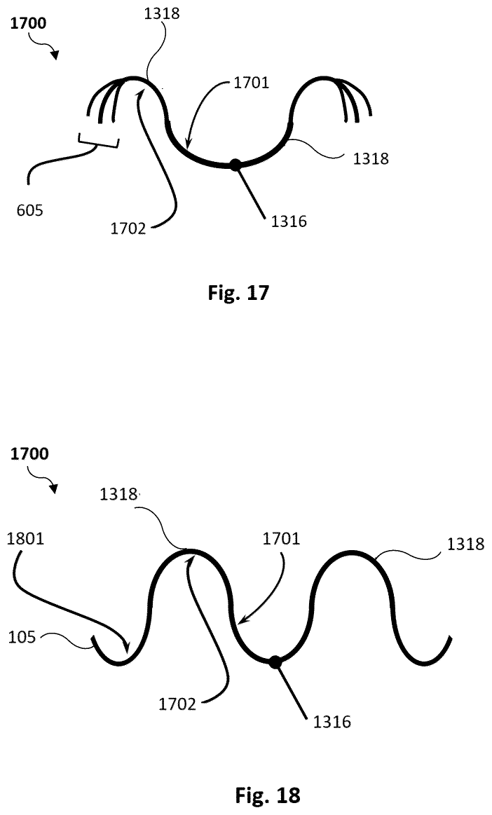

Referring now to FIGS. 17-22, arm geometries for the discs and the discs positioned on a center core, according to various embodiments, are illustrated. The arms 1318 can flex and stabilize the lumen.

FIG. 17 illustrates a flexible arm geometry having flared, or split, ends 605 on each of the arms 1618, to increase the contact points that anchor the device to the interior of the lumen. The curves 1701, 1702 in the arms 1318 are flexible so that the ends of the arms can contact the interior lumen wall, but are able to flex if the pressure reaches a threshold pressure. The arms 1318 can have a flexural modulus that permits bending and/or flexing of the arms to adjust to the various size lumens and apply sufficient force to stabilize the lumen and anchor the device, without damaging the lumen wall. Each arm illustrated in FIG. 17 has a first radius of curvature 1701 and a second radius of curvature 1702, along which the arms flex.

FIG. 18 depicts another exemplary embodiment of a flexible arm geometry that can be positioned on a center core. The arms 1318 can have a flexural modulus that permits bending and/or flexing of the arms to adjust to the various size lumens and apply sufficient force to stabilize the lumen and anchor the device, without damaging the lumen wall. Each arm illustrated in FIG. 18 has a first radius of curvature 1701, a second radius of curvature 1702, and a third radius of curvature 1801.

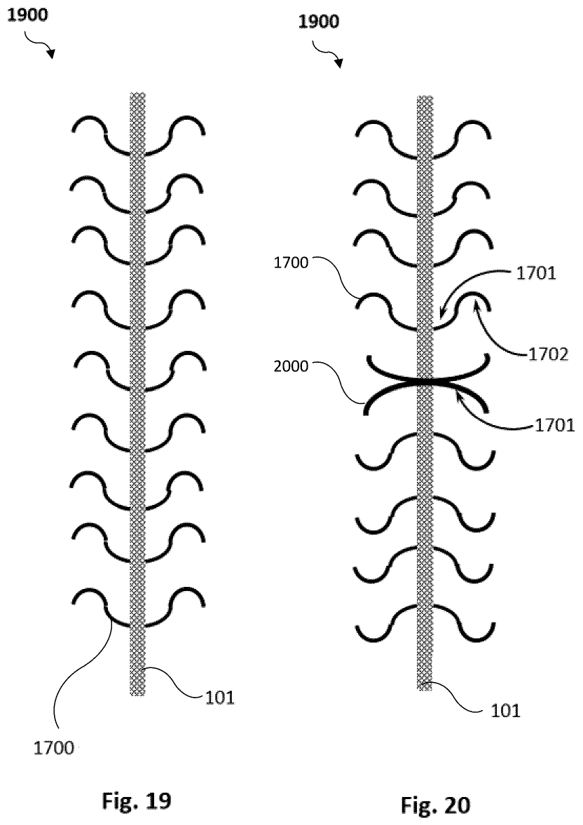

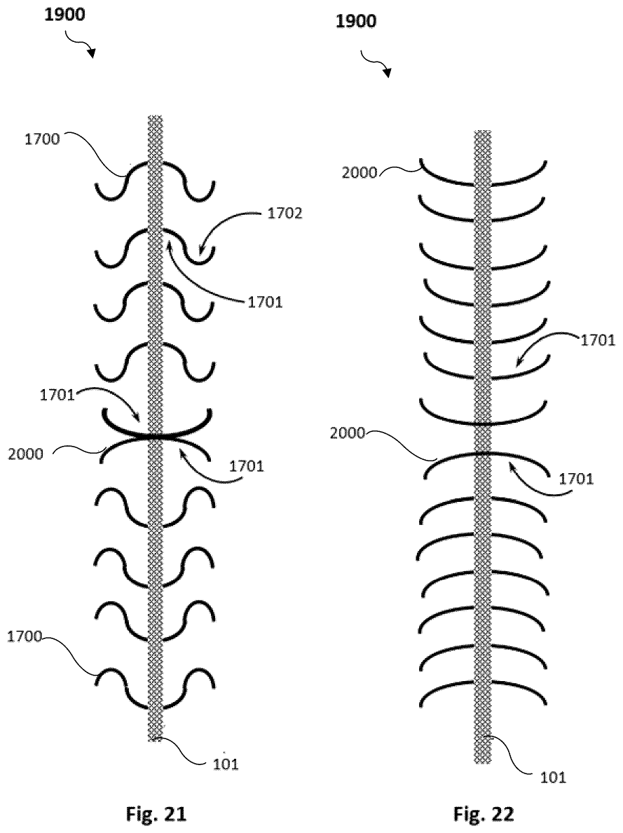

Referring to FIGS. 19-22, schematics of the various embodiments of flexible arms on a center core are illustrated. In FIGS. 19 and 20, the flexible arms of FIG. 18 are illustrated. FIG. 19 illustrates each arm flexing the same directions as the other arms on the device. FIG. 20 illustrates an embodiment where some of the arms flex in a first direction, and some of the arms flex in a second direction. The arms in the center of FIG. 20 are each flexed with only one radius of curvature. Any combination of arms can be used on the center core. FIG. 21 illustrates another exemplary embodiment, where the radius of curvature of the arms is opposite that of the arms in the device of FIG. 20. FIG. 21 illustrates a schematic of an exemplary embodiment where the arms 2000 each have one radius of curvature 1701. The arms in a first half of the device are curved in a first direction. The arms in a second half of the device can curve in a second direction that is opposite the first direction.

In FIGS. 19-22, the arms are shown in a planar view. However, the device is not limited to a planar view; the arms can rotate to create a spiral pattern, or any other pattern that permits fluid flow from one end of the device to the other while also providing support to stabilize the lumen. Further, any of the arms can have split, flared, or flattened ends as described herein.

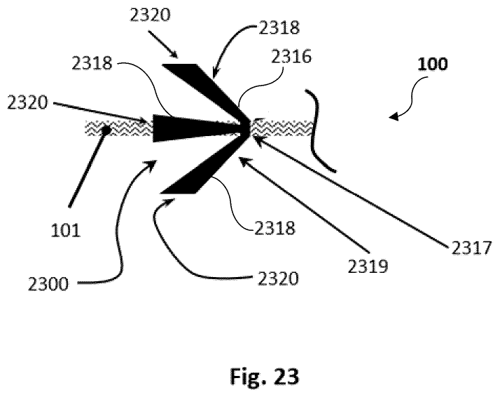

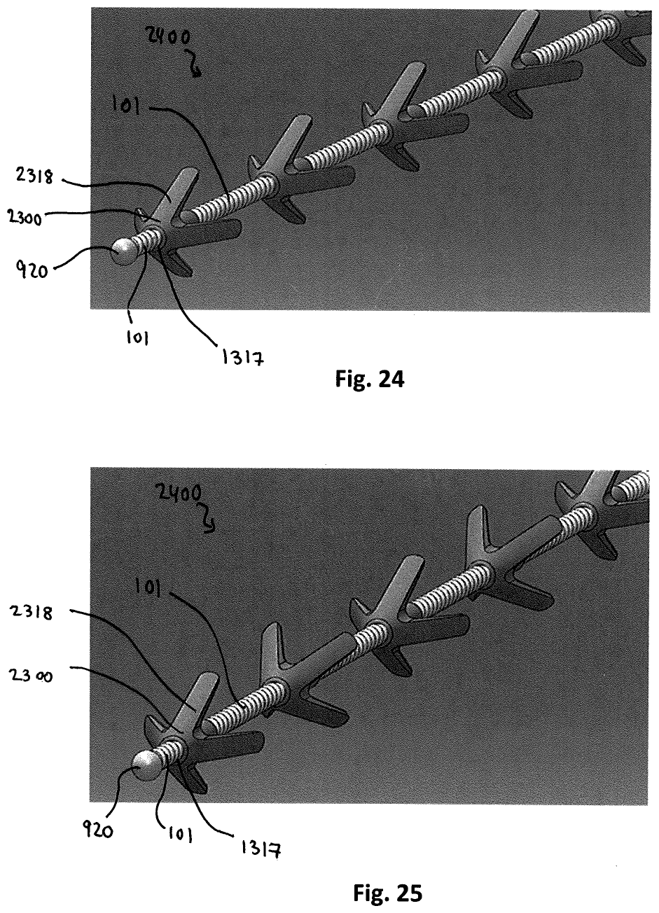

Referring now to FIG. 23, a lumen stabilizing device 100 having an umbrella-like structure 2300 on a center core 101 is illustrated. The umbrella-like structure is one embodiment of a geometry that can hold a lumen open and assist in maintaining the position of the device within the lumen such that that device does not migrate. The umbrella-like structure has a center region 2316 with a central opening 2317 for positioning the structure on a center core 101, and a plurality of arms 2318 extending outward from the center region. The arms each have a first end 2319 and a second end 2320, with the first end of each arm at the center region. The second end of each arm is a free end. When the device is implanted, the free end contacts the interior lumen wall to anchor the device. The umbrella arms extend radially outward from the core and away from the center region in a direction along the longitudinal axis of the device.

Referring now to FIGS. 24 and 25, embodiments of a lumen stabilizing device having a plurality of prongs 2318 having an umbrella-like structures on a center core 101. The prongs are similar to the arms 2318 is the umbrella-type structure illustrated in FIG. 23, and thus arms and prongs can be used synonymously throughout this description. In FIG. 24, the prongs 2318 of each structure are positioned so that they are aligned the same. That is, in an end view (not shown), they would overlap and only the end structure would be visible. In FIG. 25, the prongs 2318 of each of the umbrella-type structures are rotated from the other, so that the prongs are not aligned with each other. In FIG. 25, every other structure 2300 has its prongs aligned. In other embodiments however, the prongs 2300 can be rotated in any way to stabilize the lumen so fluid can flow from one end of the device to the other. The center core in FIGS. 24 and 25 also has a ball-shaped grasping feature at the end, to assist with placement and/or removal of the device in a lumen.

A lumen stabilizing device with an umbrella-like structure can be inserted into a lumen by a delivery catheter. The device can be positioned within the delivery catheter with the arms 1318 (or prongs 2318) in a collapsed configuration against the center core. Once the delivery catheter is positioned where the device is to be implanted, the delivery catheter can be removed, which allows the arms to flex outward. Pulling the device 2400 in a direction opposite the direction of fluid flow and peristaltic waves in the lumen, the contact between the edges of the arms and the interior lumen wall increases to assist with anchoring the device.

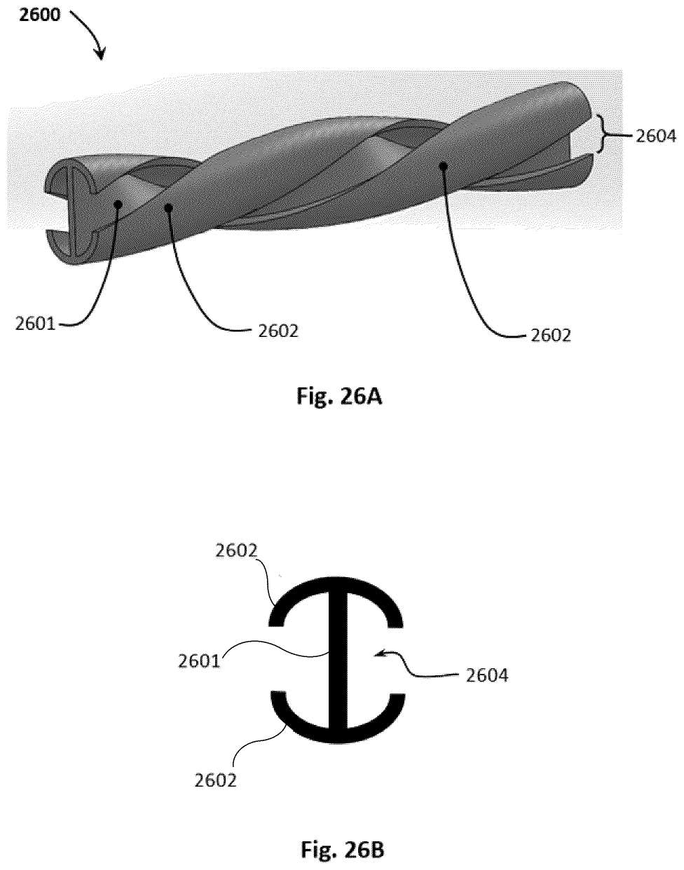

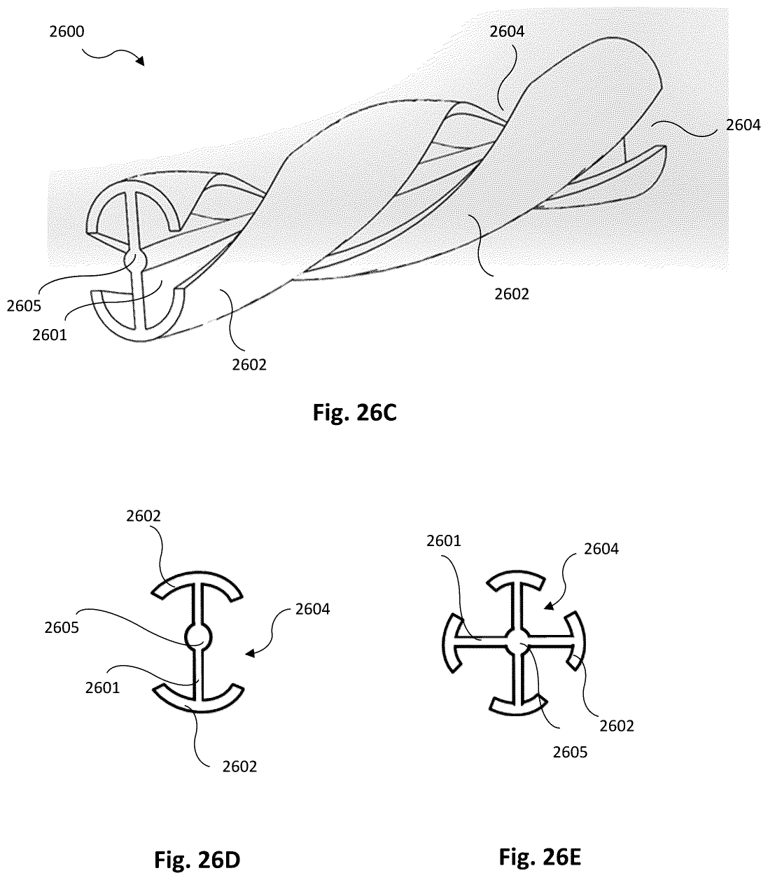

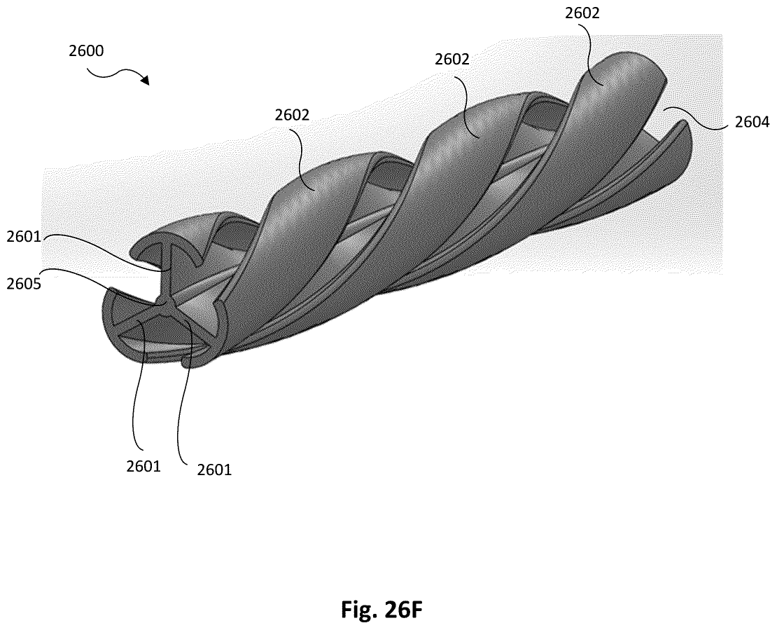

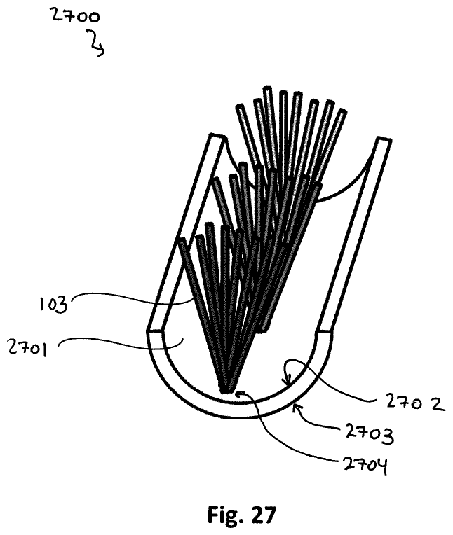

Referring now to FIGS. 26A and 26B, a lumen stabilizing device 2600 having a center beam 2601 and curved support members 2602 is illustrated. This embodiment is a variation of an I-beam structure. FIG. 26A illustrates a center beam, with first and second support members 2602. The center beam is similar to the “web” of a typical I-beam structure, and the support members are similar to the “flanges” of a typical I-beam structure. The support members 2602 are curved to substantially match the curved interior of a lumen wall. The entire device is twisted so that the center beam 2601 and the support members 2602 twist and curve such that the curved members rotate around the inside of a lumen. The volume 2604 between the curved members on either side of the center beam 2601 also rotates along the length of the device, and therefore the lumen, to create a spiral pathway. Fluid can flow through the spiral pathway on both sides of the center beam 2601 when the device is positioned in a lumen. The device 2600 can be extruded and can be made of biocompatible polymers such as HDPE, PP, TPE, PTFE, or silicone. FIG. 26B illustrates a cross-section of the device 2600 with an I-beam shape with a center beam 2601 and curved support members 2602.

Referring now to FIGS. 26C and 26D, an exemplary embodiment of a lumen stabilizing device 2600 having a center beam 2601, curved support members 2602, and a center core 2605 is illustrated. The embodiment of FIGS. 26C and 26D illustrate another variation of an I-beam structure. FIG. 26C illustrates a center beam 2601, with first and second support members 2602. The center beam is similar to the “web” of a typical I-beam structure, and the support members are similar to the “flanges” of a typical I-beam structure. The support members 2602 are curved to substantially match the curved interior of a lumen wall. There can be a center core 2605 that extends along the length of the device in the middle of the center beam 2601. The entire device is twisted so that the center beam 2601 and the support members 2602 twist and curve such that the curved members rotate around the inside of a lumen. The center core, while it can also twist, can maintain its position in the center or near the center of the device. The center core can add rigidity and/or it can be made of a material that possesses greater stiffness than the support members. The volume 2604 between the curved members on either side of the center beam 2601 also rotates along the length of the device, and therefore the lumen, to create a spiral pathway. Fluid can flow through the spiral pathway on both sides of the center beam 2601 when the device is positioned in a lumen. The device 2600 can be extruded and can be made of biocompatible polymers such as HDPE, PP, TPE, PTFE, or silicone. FIG. 26D illustrates a cross-section of the device 2600 with an I-beam shape with a center beam 2601, curved support members 2602, and a center core 2605. In FIGS. 26C and 26D, the structure can also be described as a center core 2605 having support members 2601 extend outward from the center core, and each support member can have a flange 2602 on the radially outward end of it. In this way of describing the lumen stabilizing device 2600, the center beam 2601 now refers only to a portion that extends from the center core 2605 to the flange 2602. The support members 2601 can be akin to the fins 1318 in FIG. 16A for example, and the flanges 2602 can be similar to the geometrical ends 1319 of the fins in FIG. 16A. As described below, with respect to FIGS. 26E and 26F, there can be a plurality of support members 2601 from the center core 2605, each having a flange 2602.

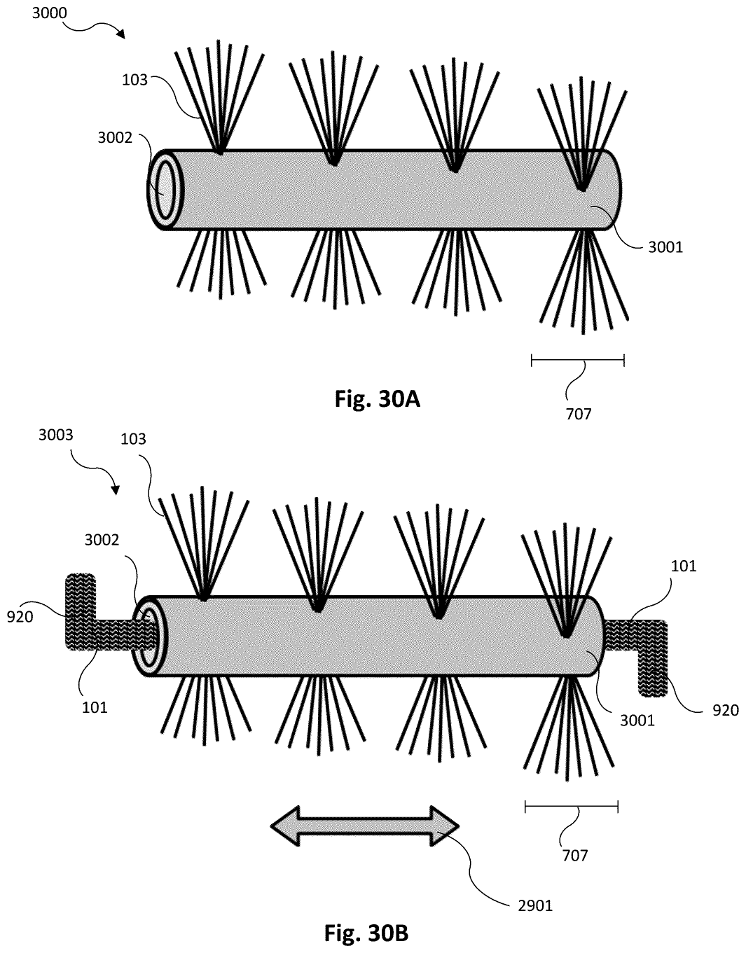

Referring now to FIGS. 26E and 26F, exemplary embodiments of lumen stabilizing devices incorporating similar features as those of FIGS. 26A-D are illustrated. FIG. 26E illustrates a cross-section of a lumen stabilizing device having a center core 2605, and four support members 2601. Each of the support members 2601 can have a flange 2602 on the end that is positioned radially outward from the center 2605. The volume 2604 between the support members permits fluid and particles to flow from one end of the device to the other. The device can be rotated as described in FIG. 26A, or it can be straight. FIG. 26F illustrates a lumen stabilizing device 2600 having a center core 2605, and three support members 2601. Each of the support members 2601 can have a flange 2602 on the end that is positioned radially outward from the center. The flanges 2602 can be curved, or they can have any of the geometries described herein. The device can be rotated as described in FIG. 26A, or it can be straight. In any of the embodiments illustrated in FIGS. 26C-26F, the center 2605 can have a hollow lumen extending through it (see FIG. 30A).

FIG. 26C depicts a perspective view of a lumen stabilizing device with a center beam and support members on opposing sides of the beam, coextruded and twisted, and having a defined center core, according to an exemplary embodiment.

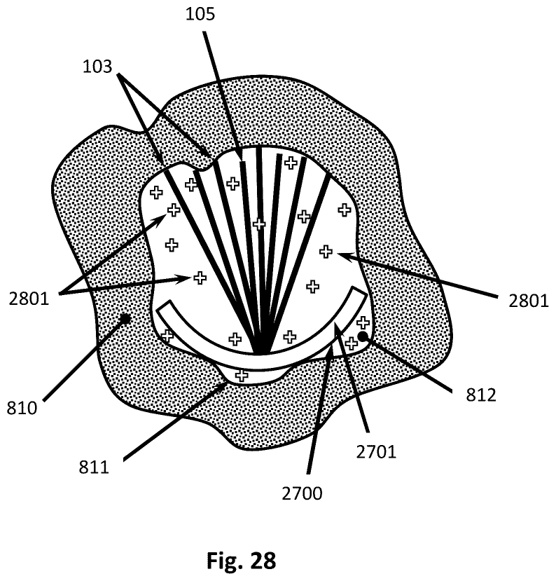

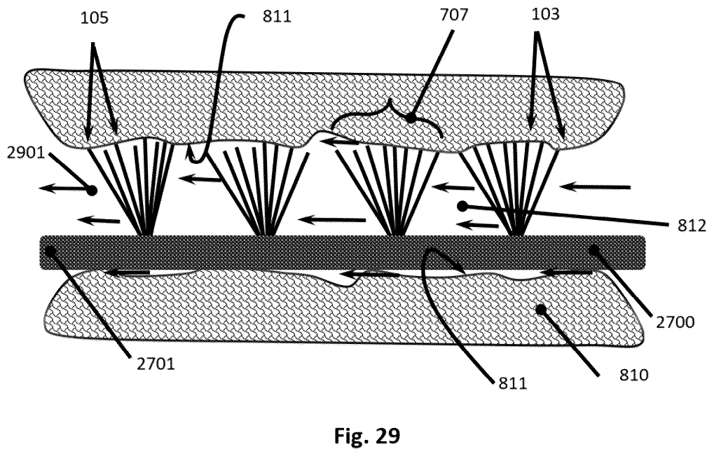

Referring now to FIG. 27, another exemplary embodiment of a lumen stabilizing device 2700 is illustrated. The device 2700 can have a substrate 2701 with an outer surface 2703 for contacting a lumen wall, and an interior surface 2702. The interior surface of the substrate can have bristles grouped in bunches as described above, extending towards a direction opposite the bristle entry points 2704 in the substrate. The bristle ends can be unadorned, and/or can be flattened, and/or flared, and/or possess any atraumatically-shaped end. The bristles can have a micro- or nano-textured surface. The space between the bristles and the substrate permit fluid flow from one end of the device to the other. The substrate can be U-shaped or a half-round shape and can be made from an extrusion of material such as polypropylene or nylon. The fluid can flow beneath and around the base, as well as through and between the bristles that can stabilize the lumen and hold it open. FIGS. 28 and 29 illustrate the flow of fluid through the lumen from one end of the device to the other. FIG. 28 illustrates a cross-section view of a lumen with an interior lumen wall 811. The interior wall of the lumen is not perfectly circular, but can have variations and an uneven surface along the length of the lumen. For example, an obstruction can alter the shape and size of at least a portion of the lumen. The device fits in the lumen, and portions of the substrate 2701 contact and support the lumen wall 811, while on the opposing side of the device, the ends 105 of the bristles 103 in the bristle bunches 707 contact and support the lumen wall 811. The fluid flow around and past all surfaces of the device is indicated by x's 2801. In FIG. 29, a side view of the lumen and device are illustrated. In this view, the direction of fluid flow is indicated by arrows 2901, past all surfaces of the device 2700, including both the substrate 2701 and the bristles 103.

Referring now to FIGS. 30A and 30B, a lumen stabilizing device 3003 made of a hollow tube with support features and a center core is illustrated. FIG. 30A illustrates the support elements and hollow tube 3001 of the device, and FIG. 30B illustrates the device 3003 having a center core 101 extend through a lumen 3002 of the hollow tube 3001. The support features can be bristles 103 and the bristles can be grouped in bunches 707. In FIGS. 30A and 30B, the bunches can rotate about the hollow tube 3001. The hollow tube can be injection molded. Instead of bristles, the hollow tube can have fins extending outward in a radial direction, or a combination of support members as described herein. The hollow tube 3001 can be positioned over a center core 101. The center core 101 can have end pieces 920 for use when positioning the lumen stabilizing device. In this exemplary embodiment, the tube can remain moveable longitudinally and/or rotationally on the center core, or it can be fixed to the core. In this embodiment, the support elements are secured to the tube, which is attached to the core, rather than being directly secured to the core. The support elements in this embodiment can move independent of the core, but remain fixed to the tube.

Referring now to FIGS. 31 and 32, an exemplary embodiment of a lumen stabilizing device 100 and a delivery system 3200 for the device 100 is illustrated. In FIG. 31, the device is similar to that illustrated in FIG. 9, but any of the exemplary embodiments described herein can be used with the delivery system illustrated in FIG. 32. The device 100 has bristle brush groupings 707 separated by gaps 708. The device 100 has atraumatic tips 920 at each end of the center core 101. Each atraumatic tip 920 can be a loop. The loop can be grasped with a grasper or other tool, in the event the lumen stabilizing device is to be removed. A ball end or other rounded, atraumatic shape can also be used.

In FIG. 32, the device 100 is positioned inside a distal portion of a delivery system 3200. The system can have an over-catheter 3201 that can be a thin-walled catheter. The over-catheter can be made of a polymer such as poly-ether-ether-ketone (PEEK). The over-catheter can have an atraumatic tip 3202 that can be shaped to accommodate the atraumatic tip 920 of the lumen stabilizing device 100. The over catheter is snugly fit over an inner catheter 3203. The inner catheter 3203 can have a lumen a drive wire 3204 and pusher tube 3205. The inner catheter can have a second lumen (not shown) for a guide wire (not shown). Optionally, the system can have any feature for incorporating a guide wire known in the art. When assembled, the lumen stabilizing device 100 is positioned inside the over-catheter 3201, and the bristles are compressed within the over-catheter to reduce the profile of the device so that it fits within the catheter for delivery purposes. When the device 100 and distal end of the delivery system is positioned in the lumen, the over-catheter is pulled back in a proximal direction and/or the drive wire and pusher tube are pushed in a distal direction. As the device is moved distally out of the over-catheter, the bristles expand from the compressed position to a position where they are extending radially outward and interacting with the interior lumen wall. Other exemplary embodiments of delivery systems can include a balloon or other dilation feature. The system 3200 can be used with other commercially available delivery systems known in the art.

Accordingly, the various embodiments are not to be limited in scope by the specific embodiments described herein. Further, although some of the embodiments have been described herein in the context of a particular implementation in a particular environment for a particular purpose, those of ordinary skill in the art should recognize that its usefulness is not limited thereto and that the various embodiments can be beneficially implemented in any number of environments for any number of purposes. Accordingly, the claims set forth below should be construed in view of the full breadth and spirit of the embodiments as disclosed herein. While the foregoing description includes many details and specificities, it is to be understood that these have been included for purposes of explanation only and are not to be interpreted as limitations of the various embodiments. Many modifications to the embodiments described above can be made without departing from the spirit and scope of this description.

What is claimed is:

1. A device for implantation in and stabilization of a lumen, comprising:
a beam extending along a length of the device, the beam having a first radial end, a second radial end, a first side, and a second side opposite the first side;
a first support member disposed along the first radial end of the beam, the first support member extending circumferentially from the first and second sides of the beam; and
a second support member disposed along the second radial end of the beam, the second support member support member extending circumferentially from the first and second sides of the beam;
wherein the support members are curved to correspond to a curved interior of a lumen wall.

2. The device of claim 1, wherein the beam is twisted along the length of the device.

3. The device of claim 1, wherein the device comprises an extruded polymer.

4. The device of claim 1, wherein fluid may flow between the support members from a first end of the device to a second end of the device.

5. The device of claim 1, further comprising a core extending the length of the device in a middle of the beam.

6. The device of claim 5, wherein the beam is twisted about the core.

7. The device of claim 5, further comprising a lumen extending through the core along the length of the device.

8. A device for stabilizing a lumen in an open position, comprising:
a beam extending along a length of the device, the beam having a first radial end, a second radial end, a first side, and a second side opposite the first side;
a first support member disposed along the first radial end of the beam, the first support member extending circumferentially from the first and second sides of the beam;
a second support member disposed along the second radial end of the beam, the second support member support member extending circumferentially from the first and second sides of the beam; and
a core extending the length of the device in a middle of the center beam;
wherein the beam is twisted around the core such that the support members rotate around the core and define spiral pathways on both sides of the beam.

9. The device of claim 8, wherein the support members are curved to correspond to a curved interior of a lumen wall.

10. The device of claim 8, wherein the device comprises an extruded polymer.

11. The device of claim 8, the beam has a rectangular cross section along the length of the device.

12. The device of claim 8, wherein the core is stiffer than the support members.

13. The device of claim 8, wherein the core is disposed in a middle of the device along the length of the device.

14. The device of claim 8, wherein the core defines a lumen extending through a center of the core.

15. A device for implantation in and stabilization of a lumen, comprising:
a core having a longitudinal axis along a length of the core;
a first support member and a second support member, the first and second support members extending radially outwardly from the core along the length of the core; and
flanges disposed at a radially outer end of each support member and extending circumferentially from the radially outer end of the support member;
wherein the device defines volumes between the flanges and the core operable to permit a flow of fluid and particles from a first end of the device to a second end of the device.

16. The device of claim 15, wherein the first and second support members are disposed on opposite sides of the core.

17. The device of claim 15, further comprising a third support member extending radially outwardly from the core along the length of the core.

18. The device of claim 15, wherein the flanges are curved.

19. The device of claim 15, wherein the flanges are straight.

20. The device of claim 15, wherein the core comprises a lumen extending through the length of the core.
